(19) United States
(12) Patent Application Publication
(10) Pub. No.: US 2018/0080061 A1
(43) Pub. Date: Mar. 22, 2018

(54) BIOSYNTHETIC LABELING AND SEPARATION OF RNA

(71) Applicant: THE REGENTS OF THE UNIVERSITY OF CALIFORNIA, Oakland, CA (US)

(72) Inventors: Michael Cleary, Mariposa, CA (US); Naoki Hida, Merced, CA (US)

(21) Appl. No.: 15/557,391

(22) PCT Filed: Mar. 18, 2016
(86) PCT No.: PCT/US16/23232

§ 371 Date: Sep. 11, 2017

Related U.S. Application Data
(60) Provisional application No. 62/137,112, filed on Mar. 23, 2015.

(51) Int. Cl. C12Q 1/68

(52) U.S. Cl. CPC C12Q 1/6806; C12Y 204/02009; C12Y 305/04001

(57) ABSTRACT

Methods are provided for differential biosynthetic labeling of RNA, including identification of cell type-specific programs of gene expression. The methods and compositions of the invention allow detection and/or purification of RNAs with precise spatial and temporal resolution. In various embodiments of the invention, the methods are applied to animal cells, including cell lines, stem cells, selected lineages of organisms, and the like.

### CROSS-REFERENCE

This application claims the benefit of U.S. Provisional Patent Application No. 62/137,112, filed Mar. 23, 2015, which application is incorporated herein by reference in its entirety.

### GOVERNMENT RIGHTS

This invention was made with Government support under contract HD076927 awarded by the National Institutes of Health. The Government has certain rights in the invention.

### BACKGROUND OF THE INVENTION

Cell type specific gene expression is a defining feature of multicellular organisms. The analysis of cell type specific transcriptomes can provide insight into the mechanisms used to generate cellular diversity, as well as help determine the underlying cause of disease. Although a few methods are available for cell type specific RNA isolation, each has constraints and researchers are often limited by their ability to isolate RNA from cell types of interest. Thus, developing new methods for cell type specific RNA isolation and detection is an important goal for genomic analysis of development and disease.

The most common methods for isolating cells for transcriptional profiling, which include fluorescence-activated cell sorting (FACS), laser capture, manual dissection, and panning, require dissociation of the targeted cells from their host tissue. While these methods are effective, they have practical and theoretical limitations. In practice, many of these methods are slow or laborious, require expensive equipment, or are not capable of isolating dispersed cell types. These methods run the risk of losing RNA in fine cellular processes (e.g., axons, dendrites, or glial processes) and can induce non-physiological changes in gene expression during the dissociation procedure.

As a response to these problems, several genetic methods have been recently developed for isolating RNA from specific cell types from within intact tissues without the need for cell dissociation (Heiman et al. 2008; He et al. 2012). These methods succeed in avoiding dissociation trauma, but they, too, have limitations. Each method only isolates a subset of cellular RNA (messenger RNA [mRNA] or microRNA [miRNA]), each requires overexpression of an endogenous mouse protein that could have deleterious effects, and each provides only limited temporal control of labeling.

It was previously shown that the Toxoplasma gondii nucleotide salvage enzyme uracil phosphoribosyltransferase (UPRT) can be used to biosynthetically label newly synthesized RNA in vivo. Under natural conditions, UPRT couples ribose-5-phosphate to the N1 nitrogen of uracil to yield uridine monophosphate (UMP) which is subsequently incorporated into RNA. When the modified uracil analog 4-thiouracil (4TU) is provided to UPRT as a substrate, the resultant product is also incorporated into RNA, and this incorporation has little effect of cellular physiology. Thio-substituted nucleotides are not a natural component of nucleic acids, and the resulting thio-labeled RNA can be readily tagged and purified using commercially available reagents.

The present invention provides improvements to methods of selectively labeling and isolating mRNA from distinct cell populations.

### RELEVANT LITERATURE

Gay et al. Genes Dev. 2013 Jan. 1; 27(1):98-115; Miller et al. Nat Methods. 2009 June; 6(6):439-41; Cleary et al. Nat Biotechnol. 2005 February; 23(2):232-7; Darzynkiewicz et al. Cytometry A. 2011 May; 79(5):328-37; Guan et al. Chembiochem. 2011 Sep. 19; 12(14):2184-90; Johnson et al. Cancer Gene Ther. 2011 August; 18(8):533-42; O'Brien et al. Hum Gene Ther. 2006 May; 17(5):518-30; Miyagi et al. J Gene Med. 2003 January; 5(1):30-7.

Multisite cloning vectors and the use thereof in recombination are disclosed, for example, in Hartley et al. (2000) Genome research 10:1788-1795; Sasaki et al. (2005) J. Biotechnology; Sasaki et al. (2004) J. Biotechnology 107:233-243.

### SUMMARY OF THE INVENTION

Methods are provided for differential biosynthetic labeling of RNA, including identification of cell type-specific programs of gene expression. The methods and compositions of the invention allow detection and/or purification of RNAs with precise spatial and temporal resolution. In various embodiments of the invention, the methods are applied to animal cells, including cell lines, stem cells, selected lineages of organisms, and the like. The biosynthetically labeled RNA thus produced can be analyzed and/or isolated by any convenient method, including sequencing, hybridization, affinity selection, and the like.

In the methods of the invention, a nucleoside analog that provides a tag for quantitative separation of the RNA away from unlabelled RNA, or for addition of a second moiety that provides for a detectable label, is selectively introduced into RNA. Using this technique, RNA so labeled can be efficiently and specifically isolated away from all other RNA and analyzed. The RNA thus labeled can be used to quantitate newly synthesized RNA independent of any pre-existing RNA, and can rapidly and sensitively detect changes that occur when genes are switched on or off. The methods also allow distinction between mRNA made in different cells within a given tissue or sample, e.g. cells that have different functions, are infected vs. uninfected, or are from different host origins, for example in animals that are chimeric for a transgene. The methods of the invention are also useful for purification of specifically labeled RNA. The reactive moiety permits determination of interaction between RNA and proteins, nucleic acids, and other molecules, e.g. by cross-linking of the moiety to nearby atoms.

Labeling is performed by introducing into a cell or lineage of interest genetic sequences encoding each the nucleoside-salvage enzymes: cytosine deaminase (CD) and uracil phosphoribosyltransferase (UPRT); or a fusion protein that combines the two activities. The enzymes may be from the same or different source organism. In some embodiments the enzymes are S. cerevisiae enzymes. In some embodiments, the enzymes are provided in one or two expression cassette regulated by an activity of interest, e.g. activated by Gal4/UAS; cre recombinase, tetracycline, acdysone, etc. The sequential activity of CD and UPRT enzymes converts cytosine to uridine monophosphate, which is subsequently incorporated into RNA. Animals lack cytosine deaminase activity and have very limited UPRT activity. Thus, providing a modified nucleoside substrate for these enzymes allows tagging of nascent mRNAs at specific times and only in cells expressing the combination of CD and UPRT. In some embodiments the nucleoside substrate is 5-ethynyl-cytosine (4-amino-5-ethynylpyrimidine-2(1H)-one).

In one embodiment of the invention, the purine or pyrimidine analog includes an alkyne moiety, thereby providing a reactive moiety not normally present in nucleic acids. Other such moieties might include sulfonyl, nitro, chloro, bromo, fluoro, sulfamino, azide, etc. Preferably the analog is not toxic to the cell. The analog may be a pyrimidine analog, e.g. a cytosine analog. The alkyne moiety can readily react with azide reagents using known “click” chemistry methods and reagents, allowing introduction of groups useful in separation and detection, e.g. haptens or molecules having known high affinity ligands, e.g. biotin, digoxigenin, etc.; specific labels, e.g. fluorescein, Cy3, Cy5, etc.; direct linking to substrate surfaces, e.g. capillaries, magnetic beads, microspheres; and the like.

In some embodiments of the invention, kits are provided for detection and/or purification of RNAs with precise spatial and temporal resolution. Such kits typically comprise a nucleoside analog suitable for labeling, e.g. 5-ethynyl-cytosine in a dosage form suitable for administration to animals or cell culture. Kits may further comprise one or both of the enzymes utilized in the methods of the invention, e.g. cytosine deaminase (CD) and uracil phosphoribosyltransferase (UPRT). The enzymes may be provided separately on vectors or expression cassettes, or may be provided in a single vector, or as a fusion protein. The enzymes may be provided on a vector suitable for recombination into animal cells, e.g. using the lambda recombinase system.

Kits may further comprise a labeling reagent, e.g. biotin-azide, an azide-fluorochrome conjugate, etc. Kits may further comprise streptavidin conjugate for RNA purification, e.g. streptavidin conjugated to a magnetic particle. For RNA analysis, a kit may further comprise, without limitation, primers and reagents for RNA sequencing.

### BRIEF DESCRIPTION OF THE DRAWINGS

FIG. 1 is a schematic illustrating an exemplary pathway for producing biosynthetically labeled RNA.

### DESCRIPTION OF THE SPECIFIC EMBODIMENTS

Methods are provided for differential biosynthetic labeling of RNA, including identification of cell type-specific programs of gene expression. The methods and compositions of the invention allow detection and/or purification of RNAs with precise spatial and temporal resolution. In various embodiments of the invention, the methods are applied to animal cells, including cell lines, stem cells, selected lineages of organisms, and the like.

Enzymes.

Enzymes of interest for use in the methods of the invention are the nucleotide salvage pathway enzymes cytosine deaminase (CD) and uracil phosphoribosyltransferase (UPRT). These enzymes have the EC classifications 2.4.2.9 (uracil phosphoribosyltransferase) and 3.5.4.1 (cytosine deaminase). Enzymes with these activities can be obtained from various prokaryotic and eukaryotic organisms, including prokaryotes, such as Escherichia coli; Bacillus subtilis; Bacillus caldolyticus; Helicobacter pylori; Lactococcus lactis; Methanobacterium thermoautotrophicum; Mycoplasma pneumoniae; Mycobacterium bovis BCG; Streptococcus salivarius; Streptomyces tendae; Sulfolobus shibatae; and protozoans, e.g. Crithidia luciliae; Giardia intestinalis; Giardia lamblia; Plasmodium sp.; Tritrichomonas foetus; etc. and yeast, e.g. Candida albicans; Candida glabrata; Saccharomyces cerevisiae; etc.

Exemplary enzymes include, without limitation, uracil phosphoribosyltransferase from Saccharomyces cerevisiae, the sequence of which is publicly available at Genbank, accession number NP_011996, herein specifically incorporated by reference, as described in Johnston et al. Science 265 (5181), 2077-2082 (1994).

An exemplary cytosine deaminase is from Saccharomyces cerevisiae, the sequence of which is publicly available at Genbank, accession number AAB67713, herein specifically incorporated by reference. The yeast enzyme is a homodimer with individual subunits comprised of 158 residues, corresponding to a mass of 35 kDa per functional oligomer. In other embodiments, a fusion protein of the CD and UPRT enzyme can be used, for example as commercially available from Invivogen, (catalog # psetz-fcyfur).

The suitability of a candidate enzyme for use in the methods of the invention may be empirically determined, using methods known in the art. Candidate enzymes can be selected based on similarity of amino acid sequence to a known phosphoribosyl transferases or thymidine kinases, by detection of biological activity, by selection from known enzymes, etc. The activity of the enzyme in transferring a purine or pyrimidine analog of interest into a nucleotide is readily determined using known assays, for example as described in the examples provided herein; as described by Iltzsch and Tankersley (1994) Biochem Pharmacol. 48(4):781-92; and the like.

The methods of the invention also include the use of a “variant” enzyme, which means a biologically active polypeptide as defined above, having less than 100% sequence identity with a naturally occurring enzyme. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Such variant polypeptides are functional, in that they retain the biological and/or biochemical activity of interest.

In various embodiments the enzymes or fusion protein are provided on one or two expression vectors. For example, a standard plasmid with a ubiquitous promoter can be used. Alternatively an inducible promoter can be used, e.g. in a “Q-system” as described by Riabinina et al. (2015) Nature Methods 12(3):219-222, herein specifically incorporated by reference. To make cell type-specific expression in mammalian cell lines, the enzymes may be provided on a vector suitable for recombination, e.g. Multisite gateway vectors, and the like as known in the art. Alternative a BAC construct can be provided with the enzymes placed under the control of a desired enhancer/promoter.

Substrate.

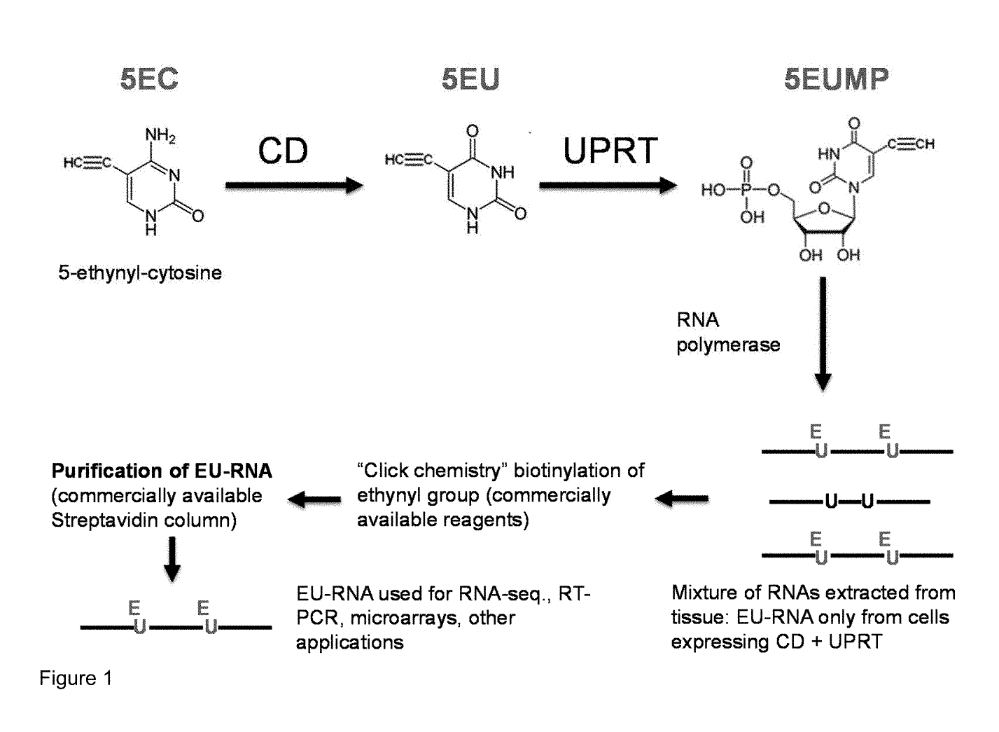

The methods of the invention utilize a pathway of enzymes, as shown in FIG. 1. The pyrimidine salvage pathway enables organisms to utilize exogenous pyrimidine bases and nucleosides, which are not intermediates in de novo pyrimidine synthesis. Cytosine deaminase (CD; EC 3.5.4.1) catalyzes the deamination of cytosine to uracil and ammonia. Cytosine deaminase is found in bacteria and fungi, where it plays an important role in pyrimidine salvage, but is not present in mammalian cells.

Various substrates of CD are known in the art, e.g. cytosine itself, 2-hydroxypyrimidine, 5-fluorocytosine, etc. Preferred substrates are not themselves toxic to animal cells, and are not converted to toxic products. In some embodiments the substrate is 5-ethynyl-cytosine (5EC). The alkyne group of 5EC provides a convenient moiety to attachment of functional groups through click chemistry reactions.

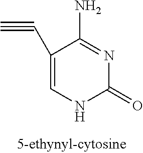

5-ethynyl-cytosine has been described in the art. See, for example, Barr et al. (1978) J. Chem. Soc., Perkin Trans. 1:1263-1267. 5EC can be synthesized from 5-iodo-2′-deoxycytosine by Sonogashira coupling of trimethylsilyacetylene. The Pd₀-mediated coupling reaction between alkynes and unprotected nucleosides has been used to prepare similar compounds.

Labeling Systems.

As noted above, the alkyne group of 5EC provides a reactant group for CLICK chemistry reactions (see Click Chemistry: Diverse Chemical Function from a Few Good Reactions Hartmuth C. Kolb, M. G. Finn, K. Barry Sharpless Angewandte Chemie International Edition Volume 40, 2001, P. 2004, herein specifically incorporated by reference).

Other chemistries such as the copper-free variant of this reaction (which uses a strained alkyne moiety), oxime formation between an acetyl group and an amino-oxy moiety, and a modified Staudinger ligation between an azide and a phosphine are also of interest.

Labeling compounds are known in the art and commercially available for modifying alkynes, including for example biotin azide (PEG4 carboxamide-6-azidohexanyl biotin); azide conjugates of fluorophores; azide conjugates of magnetic beads; azide conjugates of amino acids, including conjugates of polypeptides, and the like. Where the reaction is performed with a copper catalyst, suitable copper (II) sulfate (CuSO₄) may be provided.

Expression Construct:

The salvage pathway enzymes are provided in an expression construct. Each of the enzymes may be provided in a single construct, or in individual constructs. The DNA encoding the enzyme may be obtained from a cDNA library prepared from tissue expressing the mRNA; from a genomic library; by oligonucleotide synthesis; by PCR amplification using specific or consensus primers, and the like. As described above, there are many nucleotide salvage enzyme genetic sequences known in the art. Libraries may be screened with probes designed to identify the gene of interest or the protein encoded by it. Screening the cDNA or genomic library with the selected probe may be conducted using standard procedures as described in Sambrook et at, Molecular Cloning: A Laboratory Manual (New York: Cold Spring Harbor Laboratory Press, 1989). An alternative means to isolate the gene encoding UPRT is to use PCR methodology.

Amino acid sequence variants of enzymes are prepared by introducing appropriate nucleotide changes into the encoding DNA, or by synthesis of the desired protein. Such variants represent insertions, substitutions, and/or specified deletions of, residues within or at one or both of the ends of the amino acid sequence of a naturally occurring UPRT. Preferably, these variants represent insertions and/or substitutions within or at one or both ends of the mature sequence, and/or insertions, substitutions and/or specified deletions within or at one or both of the termini. Any combination of insertion, substitution, and/or specified deletion is made to arrive at the final construct, provided that the final construct possesses the desired biological activity.

The nucleic acid encoding the enzyme of interest can be inserted into an integrating or replicable vector for expression. Many such vectors are available, including episomal vectors, integrating vectors, viral vectors, etc. The vector components generally include, but are not limited to, one or more of the following: an origin of replication, one or more marker genes, an enhancer element, a promoter, and a transcription termination sequence.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors contain a promoter that is recognized by the cell of interest, and is operably linked to the enzyme coding sequence. Promoters are untranslated sequences located upstream (5′) to the start codon of a structural gene (generally within about 100 to 1000 bp) that control the transcription and translation of a particular nucleic acid sequence to which they are operably linked. Promoters may be inducible or constitutive, where inducible promoters broadly include promoters induced by a variety of developmental and environmental cues. Inducible promoters are promoters that initiate increased levels of transcription from DNA under their control in response to some change in conditions, e.g., the presence or absence of a nutrient, factor, developmental state, etc. A large number of promoters recognized by a variety of cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment.

The promoter used may be regulated by a pathway of interest, e.g. by the presence of a signaling molecule; tissue-specific; cell type-specific promoter; etc. For example, the promoter can be one designed to substantially specify expression within a specific tissue. Exemplary tissue-specific or cell-specific promoters include, but are not limited to, myosin heavy chain promoter for muscle specific expression, Madsen et al. (1998) Circ Res 82(8):908-917; lysosomal acid lipase promoter, Du et al. (1998) Gene 208(2):285-295; pancreatic expression using the amylase promoter, Dematteo et al. (1997) J Surg Res 72(2):155-161; cardiac-specific overexpression, Kubota et al. (1997) Circ Res 81(4):627-635; folylpoly-gamma-glutamate synthetase promoter, Freemantle et al. (1997) J Biol Chem 272(40):25373-25379; tissue specific expression using neural restrictive silencer element, Kallunki et al. (1997) J Cell Biol 138(6):1343-1354, placenta specific expression using the HGH promoter, Nogues et al. (1997) Endocrinology 138(8):3222-3227, expression during pregnancy using the prolactin promoter, Schuler et al. (1997) Endocrinology 138(8):3187-3194, tissue specific expression using the alpha1(VI) collagen promoter, Braghetta et al. (1997) Eur J Biochem 247(1):200-208; B cell specific expression, Lennon et al. (1997) Immunogenetics 45(4):266-273; hypoxia induced expression, Gupta et al. (1996) Nucleic Acids Res 24(23):4768-4774; endothelium specific expression, Ronicke et al. (1996) Circ Res 79(2):277-285, the keratin promoters (e.g., human keratin 14 promoter (Wang et al. 1997 Proc Natl Acad Sci US 94:219-26); bovine cytokeratin gene promoters, BKIII and BKVI (Alexander et al. 1995 Hum Mol Genet 4:993-9); keratin 10 gene promoter (Bailleul et al. 1990 Cell 62:697-708); and tyrosinase promoters (specific for melanocytes)). Epidermal-specific promoters are reviewed in Fuchs et al. 1994 Princess Takamatsu Symp 24:290-302).

The expression can also be regulated by use of a site specific recombinase e.g. cre recombinase, FLP recombinase, pSR1 recombinase, etc. For example, a transcriptional inhibitor can be placed between two or more recombination sites. Induction of the recombinase will induce recombination between the sites, thereby deleting the inhibitor. The term “heterologous recombination site” is meant to encompass any introduced genetic sequence that facilitates site-specific recombination. In general, such sites facilitate recombination by interaction of a specific enzyme with two such sites. Exemplary heterologous recombination sites include, but are not necessarily limited to, lox sequences; recombination mediated by Cre enzyme; frt sequences (Golic et al. (1989) Cell 59:499-509; O'Gorman et al. (1991) Science 251:1351-5; recombination mediated by the FLP recombinase), the recognition sequences for the pSR1 recombinase of Zygosaccharomyces rouxii (Matsuzaki et al. (1990) J. Bacteriol. 172:610-8), and the like. A lox site is a nucleotide sequence at which the gene product of the cre gene, catalyzes site-specific recombination. A particularly preferred lox site is a loxP site. The sequence of loxP, which is 34 bp in length, is known and can be produced synthetically or can be isolated from bacteriophage P1 by methods known in the art (see, e.g. Hoess et al. (1982) Proc. Natl. Acad. Sci. USA 79:3398). Other suitable lox sites include loxB, loxL, and loxR, which can be isolated from E. coli (Hoess et al. (1982) Proc. Natl. Acad. Sci. USA 22:3398).

Construction of suitable vectors containing one or more of the above-listed components employs standard ligation techniques. Isolated vectors or DNA fragments are cleaved, tailored, and re-ligated in the form desired to generate the vectors required. For analysis to confirm correct sequences in plasmids constructed, the ligation mixtures are used to transform host cells, and successful transformants selected by ampicillin or tetracycline resistance where appropriate. Vectors from the transformants are prepared, analyzed by restriction endonuclease digestion, and/or sequenced.

Episomal expression vectors may provide for the transient expression in mammalian cells. In general, transient expression involves the use of an expression vector that is able to replicate efficiently in a host cell, such that the host cell accumulates many copies of the expression vector and, in turn, synthesizes high levels of a desired polypeptide encoded by the expression vector.

Viral vectors of interest include, without limitation, retroviral vectors (e.g. derived from MoMLV, MSCV, SFFV, MPSV, SNV etc), lentiviral vectors (e.g. derived from HIV-1, HIV-2, SIV, BIV, FIV etc.), adeno-associated virus (AAV) vectors, adenoviral vectors (e.g. derived from Ad5 virus), SV40-based vectors, Herpes Simplex Virus (HSV)-based vectors etc. A vector construct may coordinately express the enzyme of interest and a marker gene such that expression of the marker gene can be used as an indicator for the expression of the enzyme of interest, as well as for analysis of gene transfer efficiency. This can be achieved by linking the test and a marker gene with an internal ribosomal entry site (IRES) sequence and expressing both genes from a single bi-cistronic mRNA. IRES sequence could be from a virus (e.g. EMCV, FMDV etc) or a cellular gene (e.g. eIF4G, BiP, Kv1.4 etc). The examples of marker genes include drug resistance genes (neo, dhfr, hprt, gpt, bleo, puro etc) enzymes (β-galactosidase, alkaline phosphatase, etc.) fluorescent genes (e.g. GFP, RFP, BFP, YFP) or surface markers (e.g. CD24, NGFr, Lyt-2 etc). A preferred marker gene is biologically inactive and can be detected by standard immunological methods. Alternatively, an “epitope tag” could be added to the test gene for detection of protein expression. Examples of such “epitope tags” are c-myc and FLAG (Stratagene).

Cells of interest are transfected or transformed with the above-described expression vectors. The genetic construct may be introduced into tissues or host cells by any number of routes, including calcium phosphate transfection, viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into cells. After introduction into the cell, the coding sequences may integrate into the host DNA, or be maintained as a replicable vector.

The transformed or transfected cells are cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

Cells of Interest.

The methods of the present invention can employ naturally occurring cells and cell populations, genetically engineered cell lines, cells derived from transgenic animals, primary cells, normal and transformed cell lines, transduced cells and cultured cells, etc. Suitable cells include bacterial, fungal, protistan, plant and animal cells; e.g. avian; insect; reptilian; amphibian; mammalian; e.g. human, simian, rodent, etc. In one embodiment of the invention, the cells are mammalian cells; and may include complex mixtures of mammalian cells, i.e. where two or more cell types having distinguishable phenotypes are present. Examples of complex cell populations include naturally occurring tissues, for example blood, liver, pancreas, neural tissue, bone marrow, skin, and the like.

In addition, cells that have been genetically altered, e.g. by transfection or transduction with recombinant genes or by antisense technology, to provide a gain or loss of genetic function, may be utilized with the invention. Methods for generating genetically modified cells are known in the art, see for example “Current Protocols in Molecular Biology”, Ausubel et al., eds, John Wley & Sons, New York, N.Y., 2000. The genetic alteration may be a knock-out, usually where homologous recombination results in a deletion that knocks out expression of a targeted gene; or a knock-in, where a genetic sequence not normally present in the cell is stably introduced.

The expression vector can be used to generate transgenic organisms where the nucleic acid construct is randomly integrated into the genome. Vectors for stable integration include plasmids, retroviruses and other viruses, YACs, and the like. The modified cells or animals are useful in the study of gene function and regulation. For example, the enzyme of interest can be operably linked to a developmentally regulated promoter, and biosynthetically labeled mRNA used to study the regulation of gene expression, and analyze the expression profile of specific cells. Alternatively, the enzyme of interest can be regulated by a tissue specific promoter, or a promoter regulated in response to stimuli, e.g. neuronal signaling; antigen stimulation; hormone activation; exposure to toxins; etc.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected. The chimeric animals are screened for the presence of the modified gene and males and females having the modification can be mated to produce homozygous progeny or used as heterozygotes. The transgenic organism may be plants, fungus, protest, animal, etc., particularly any non-human mammal, such as laboratory animals, domestic animals, etc. The transgenic organism may be used in functional studies, drug screening, etc.

Biosynthetic Labeling.

The cells, tissue, or animal of interest is contacted with the cytosine analog to provide for temporal specificity of labeling. Where the enzyme is operably linked to a regulated (inducible) promoter, the analog may be present in the medium, feed, etc., prior to induction and biosynthetic labeling. Where the enzyme is operably linked to a constitutive promoter, the analog will be added at the time biosynthetic labeling is to commence.

The cytosine analog will be present in culture medium at a concentration of at least about 0.1 μM, usually at least about 1 μM, more usually at least about 5 μM, and not more than about 10 mM, usually not more than about 5 mM, and more usually not more than about 2.5 mM. Where the purine or pyrimidine analog is being provided to an animal, e.g. in drinking water, food, etc., the concentration will be appropriately increased to allow for losses and reduced bioavailability. The analog may be injected into an animal, e.g. by i.p. injection, at a dose of from about 10 mg/kg body weight; about 50 mg/kg body weight, about 100 mg/kg body weight, about 500 mg/kg body weight, about 750 mg/kg body weight, about 1000 mg/kg body weight. Label can be detected after about 1 hour, about 2 hours, about 3 hours, about 4 hours after injection, and tissues can be harvested at any convenient time after detectable label is incorporated, for example after about 2 hours, after about 4 hours, after about 6 hours, after about 8 hours, and the like.

In some analyses, the cytosine analog is provided as a pulse-chase, where the initial exposure to the analog is followed by exposure to a high concentration of cytosine, thereby providing a defined period of time when the RNA is biosynthetically labeled.

RNA is obtained from the cells by conventional methods. It is not necessary to separate the cells of interest from adjacent cells, although crude separation (e.g. surgical excision of a tissue) can facilitate subsequent manipulations. Solid tissue can be homogenized or otherwise broken apart, although it is not necessary. The cells are lysed to produce a suspension of RNA. Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993)).

The resulting RNA preparation will include RNA comprising the cytosine analog (converted to uracil), and will usually include unlabeled RNA. The labeled RNA can be separated from unlabeled RNA, or can be differentially tagged with a detectable label, e.g. a fluorescent label, etc., for further use.

Conveniently, a reactive moiety on the cytosine analog, usually an alkyne moiety, is reacted to form a covalent bond to a label moiety, where the tag group is a hapten or small molecule binding partner, fluorochrome, etc., e.g. digoxin, digoxigenin, FITC, dinitrophenyl, nitrophenyl, biotin, etc, or detectable label.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads, fluorescent dyes, radiolabels (e.g., ³H, ¹²⁵I, ³⁵S, ¹⁴C, or ³²P), enzymes (e.g. horseradish peroxidase, alkaline phosphatase and others commonly used in an ELISA), and colorimetric labels such as colloidal gold (e.g. gold particles in the 40-80 nm diameter size range) or colored glass or plastic (e.g. polystyrene, polypropylene, latex, etc.) beads.

A wide variety of fluorescers can be employed either alone or, alternatively, in conjunction with quencher molecules. Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene: 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4(3′pyrenyl)butyrate; d-3-aminodesoxy-equilenin; 12-(9′anthroyl)stearate; 2-methylanthracene; 9-vinylanthracene; 2,2′(vinylene-p-phenylene)bisbenzoxazole; p-bis[2-(4-methyl-5-phenyl-oxazolyl)]benzene; 6-dimethylamino-1,2-benzophenazin; retinol; bis(3′-aminopyridinium) 1,10-decandiyl diiodide; sulfonaphthylhydrazone of hellibrienin; chlorotetracycline; N(7-dimethylamino-4-methyl-2-oxo-3-chromenyl)maleimide; N-[p-(2-benzimidazolyl)-phenyl]maleimide; N-(4-fluoranthyl)maleimide; bis(homovanillic acid); resazarin; 4-chloro-7-nitro-2,1,3benzooxadiazole; merocyanine 540; resorufin; rose bengal; and 2,4-di phenyl-3(2H)-furanone. Specific fluorochromes of interest include fluorescein isothiocyanate (FITC), rhodamine, Texas Red, phycoerythrin, allophycocyanin, 6-carboxyfluorescein (6-FAM), 2′,7′-dimethoxy-4′,5-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA). Cyanine dyes are of particular interest as a detectable label. Cyanine dyes are synthetic dyes in which a nitrogen and part of a conjugated chain form part of a heterocyclic system, such as imidazole, pyridine, pyrrole, quinoline and thiazoles; including Cy3 and Cy5, which are widely used as labels. Such directly labeled RNA can be used in hybridization analysis without further manipulation.

The use of biotin is of particular interest. Biotin is a vitamin widely used in biotechnology for its ability to bind with extremely high affinity to avidin, streptavidin, neutravidin, captavidin; etc., herein generically referred to as avidins. Avidins usually each bind four biotins per molecule with high affinity and selectivity, although monomeric derivatives may also find use. Dissociation of biotin from streptavidin is reported to be about 30 times faster than dissociation of biotin from avidin. Their multiple binding sites permit a number of techniques in which unlabeled avidin, streptavidin or NeutrAvidin biotin-binding protein can be used to bridge two biotinylated reagents. Biotin can be conjugated through various chemistries to molecules of interest.

Examples of biotin reagents that will react to form covalent bonds to a thiol moiety include commercially available reagents; e.g. maleimido-biotin; maleimido-Ic-biotin; n-biotinyl-n-(3-maleimidopropionyl)-1-lysine; maleimido-peo3-biotin; HPDP-biotin (n-(6-(biotininamido)hexyl)-3′-(2′-pyridylthio) propionate); iodoacetyl-biotin (n-iodoacetyl-n-biotinylhexylenediamine); and the like. HPDP is of special interest as the disulfide linkage that it forms with the sulfhydryl is readily broken by reduction with agents such as dithiothreitol, 2-mercaptoethanol, etc. and so the original material can be restored to its original form, free of the tag, after purification.

Biotinylated RNA can be separated by affinity chromatography with a biotin binding partner, e.g. avidin, streptavidin, neutravidin; etc.; and used for sequencing, or can combined with a labeled biotin binding partner, e.g. Cy5-avidin; Cy3-avidin; and for purposes of, for example, in situ hybridization, can be combined with a radiolabeled or heavy metal labeled binding partner.

Biotin binding conjugates are extensively used as secondary detection reagents in microarrays, blot analysis, and the like. The biotinylated RNA is bound to a blot, array, cell section, etc. Detection is mediated by reagents including fluorochrome labeled avidins, enzyme-conjugated avidins plus a fluorogenic, chromogenic, or chemiluminescent substrate. Fluorescent avidin and streptavidin are extensively used in DNA hybridization techniques. Avidins can also be used as labels when conjugated to fluorescent polystyrene microspheres. Nanogold and colloidal gold conjugates find use as a label in light microscopy, and electron microscopy applications.

The use of enzyme-amplified immunodetection is a well-established standard technique. Most frequently, the enzymes of choice are horseradish peroxidase, alkaline phosphatase and Escherichia coli β-galactosidase because of their high turnover rate, stability, ease of conjugation and relatively low cost. Diaminobenzidine (DAB) can be used as a substrate with HRP, which generates a brown-colored polymeric oxidation product localized at HRP-labeled sites. The DAB reaction product can be visualized directly by bright-field light microscopy or, following osmication, by electron microscopy. Alternative substrates include fluorogenic, chromogenic and chemiluminescent substrates.

Where separation of the biosynthetically labeled RNA is of interest, affinity chromatography may be used. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural biospecific binding of one molecular species to separate and purify a second species from a mixture. Antibodies are commonly used in affinity chromatography.

Preferably a microsphere or matrix is used as the support for affinity chromatography. Such supports are known in the art and commercially available, and include activated supports that can be coupled to the linker molecules. For example, Affi-Gel supports, based on agarose or polyacrylamide are low pressure gels suitable for most laboratory-scale purifications with a peristaltic pump or gravity flow elution. Affi-Prep supports, based on a pressure-stable macroporous polymer, are suitable for preparative and process scale applications.

The binding partner for affinity chromatography can be any high affinity, usually non-covalent, interactor. Common binding partners are avidins, antibodies, and the like. The RNA sample is applied to the binding partner at a salt concentration that provides for specific binding, and is eluted off in a differential salt concentration, in the presence of free biotin or hapten, by reduction with dithiothreitol or other reducing agents; etc.

The separated RNA can be amplified prior to sequencing, hybridization, etc. If a quantitative result is desired, care must be taken to use a method that maintains or controls for the relative frequencies of the amplified nucleic acids. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

For example, the isolated RNA can be sequenced as described by Guan et al., supra. The RNA is isolated from unlabeled nucleic acids. Ribosomal RNA can be removed by various methods, including use of RNAse H, use of a ribo-zero kit, purification of poly(A) mRNAI use of oligo-dT to prime first-strand cDNA synthesis; etc. The RNA thus isolated is optionally fragmented. The RNA can be sequenced using conventional methods, including without limitation amplification and high throughput sequencing.

Another method of interest utilizes reverse transcriptase and a primer and a sequence encoding the phage T7 promoter to provide single stranded DNA template. A second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. This particular method is described in detail by Van Gelder et al. (1990) Proc. Natl. Acad. Sci. USA, 87:1663-1667. It will be appreciated by one of skill in the art that the direct transcription method provides an antisense (aRNA) pool.

The separated RNA may be labeled with a detectable label. The label may be incorporated by any of a number of means well known to those of skill in the art, e.g. during an amplification step, or reverse transcription step. For example, polymerase chain reaction (PCR) with labeled primers or labeled nucleotides will provide a labeled amplification product. Alternatively, a label may be added directly to the original RNA sample, or to the amplification product after the amplification is completed. Means of attaching labels to nucleic acids include, for example nick translation or end-labeling by kinasing of the nucleic acid and subsequent attachment of a nucleic acid linker joining the sample nucleic acid to a label. Suitable labels include any of those listed above.

Analysis.

The labeled or separated RNA can be used in a variety of hybridization protocols or sequencing protocols as known and widely practiced in the art. Procedures of particular interest include high throughput sequencing, hybridization to arrays of polynucleotide probes; and the like.

Hybridization of the labeled sequences is accomplished according to methods well known in the art. Hybridization can be carried out under conditions varying in stringency, preferably under conditions of high stringency, e.g. 6×SSPE, 65° C., to allow for hybridization of complementary sequences having extensive homology. High density microarrays of oligonucleotides are known in the art and are commercially available. The sequence of oligonucleotides on the array will correspond to the known target sequences of one of the genomes, as previously described. Arrays of interest may comprise at least about 10³ different sequences, at least about 10⁴ different sequences, and may comprise 10⁵ or more different sequences. The probes on the array may be oligonucleotides, e.g. from about 12 to 70 nucleotides in length, or may be larger sequences, e.g. cDNAs and fragments thereof. In a preferred embodiment, the microarrays used in the present methods are gene expression probe arrays. Such arrays comprise oligonucleotide probes derived from the sequence of open reading frames in the genome of interest. Commercially available high-density arrays containing a large number of oligonucleotide probes from genomic DNA sequence have been designed and used to monitor genome-wide gene expression, e.g. in mouse, human, etc.

For convenience, kits may be supplied which provide the necessary reagents in a convenient form and together. For example kits could be provided that include a vector containing one or both of the salvage pathway enzymes cytosine deaminase and uracil phosphoribosyltransferase, which may be provided with a promoter, or with a cassette for insertion of a promoter of interest. The enzymes can be provided on single vectors, or in a combination. Kits may further comprise reagents including a cytosine analog useful with the enzyme, e.g. 5-ethynyl-cytosine; etc. which may be provided in powder form, or in a solution at a concentration ready for use in the methods of the invention; biotin conjugated to an appropriate linker, e.g. azide-biotin; avidin labels or resins; and/or suitable buffers. Other components such as automated systems for determining and interpreting the hybridization results, software for analyzing the data, or other aids may also be included depending upon the particular protocol which is to be employed.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “an”, and “the” include plural referents unless the context clearly dictates otherwise. Thus, for example, reference to “a cell” includes a plurality of such cells and reference to “the array” includes reference to one or more arrays and equivalents thereof known to those skilled in the art, and so forth. All technical and scientific terms used herein have the same meaning as commonly understood to one of ordinary skill in the art to which this invention belongs unless clearly indicated otherwise.

All publications mentioned herein are incorporated herein by reference for the purpose of describing and disclosing, for example, the enzymes, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

1. A method of biosynthetically labeling RNA in a cell of interest, the method comprising:
contacting said cell with a non-toxic cytosine analog having a reactive moiety not normally present in RNA, wherein said cell comprises an exogenous cytosine deaminase and uracil phosphoribosyltransferase that can specifically incorporate said cytosine analog into the corresponding uracil nucleotide, and wherein said cytosine analog is incorporated into RNA synthesized by said cell.

2. The method according to claim 1, wherein sequences encoding said exogenous cytosine deaminase and uracil phosphoribosyltransferase are operably linked to a promoter that is active or can be activated in said cell.

3. The method according to claim 1, wherein said reactive moiety is an alkyne or azide.

4. The method of claim 3, wherein the analog is 5-ethynyl-cytosine.

5. The method of claim 1, further comprising the step of conjugating a tag to said reactive moiety.

6. The method according to claim 5, wherein said tag is biotin.

7. The method according to claim 6, wherein said detectable label is a fluorochrome, radiolabel, heavy metal label, or enzyme conjugate.

8. The method of claim 5, further comprising the step of analyzing the sequence or binding specificity of the biosynthetically labeled RNA.

9. The method according to claim 5, further comprising the step of binding a specific binding partner to said tag.

10. The method of claim 1, herein the cell of interest is an animal cell.

11. The method of claim 10, wherein the cell of interest is within a tissue of an animal.

12. The method of claim 1, wherein expression of the exogenous cytosine deaminase and uracil phosphoribosyltransferase is regulated by a promoter.

13. The method according to claim 12, wherein promoter is inducible.

14. The method according to claim 13, wherein said promoter is induced by the presence of a signaling molecule.

15. The method of claim 14, wherein the promoter activity is regulated by a cre recombinase.

16. The method according to claim 14, wherein said promoter is tissue specific.

17. The method according to claim 14, wherein said promoter is cell type-specific.

18. A kit for biosynthetic labeling of RNA, the kit comprising:
a cytosine analog having a reactive moiety not normally present in RNA; and
nucleic acid sequences encoding one or both of cytosine deaminase and uracil phosphoribosyltransferase.

19. The kit according to claim 18, wherein sequences encoding said one or both of cytosine deaminase and uracil phosphoribosyltransferase are operably linked to a promoter.

20. The kit according to claim 18, wherein said cytosine analog is 5-ethynyl-cytosine.

21. The kit according to claim 18, further comprising a tag molecule, which comprises a linker reactive with cytosine analog.
